(19) United States
(12) Patent Application Publication
(10) Pub. No.: US 2012/0171130 A1
(43) Pub. Date: Jul. 5, 2012

(54) Mitigation of Animal and Plant Diseases Using Bioavailable Minerals

(76) Inventor: John Wayne Kennedy, Stevensville, MD (US)

(21) Appl. No.: 11/932,260

(22) Filed: Oct. 31, 2007

Related U.S. Application Data
(63) Continuation-in-part of application No. 11/616,317, filed on Dec. 27, 2006, now abandoned.
Application No. 11/616,317 is a division of application No. 10/027,692, filed on Dec. 20, 2001, now Pat. No. 7,163,709.

(51) Int. Cl. A61K 8/18; A61K 33/30; A61K 33/34; A61K 33/32; A61K 39/00; A61K 33/38

(52) U.S. Cl. 424/49; 424/184.1; 424/618; 424/630; 424/639; 424/641; 424/646; 424/655; 424/682; 424/702

(57) ABSTRACT

A composition is able to treat plant and animal diseases and includes a therapeutically effective amount of an encapsulated ionic mineral complex and a pharmaceutically acceptable carrier. The composition may also include other inert ingredients, botanical extracts and vitamin and mineral supplements.

### CROSS-REFERENCE TO RELATED APPLICATIONS

This application is a continuation-in-part application of U.S. Non-provisional application Ser. No. 11/616,317 filed on Dec. 27, 2006 which is a Divisional Application of U.S. Non-Provisional patent application Ser. No. 10/027,692 filed on Dec. 20, 2001 which is now U.S. Pat. No. 7,163,709.

### FIELD OF THE INVENTION

The invention is related to a chemical composition and use for mitigating plant and animal diseases, and more specifically, to a composition having bioactive elements that are able to interrupt the metabolic pathway of various diseases affecting plants and animals with little to no affect on the host.

### BACKGROUND OF THE INVENTION

U.S. Pat. No. 7,163,709 (co-owned by the present inventor) describes the use of ionic mineral complexes against certain plant diseases and for use as an antimicrobial. It is also desirable to produce a mineral compounds for use against a wide variety of diseases of both plants and animals using a high proportion of bioavailable free mineral ions alone or in combination with a delivery system composed of a ligand system ensuring that the free associated ionic compounds reach the target cell to treat any diseased target cell.

### SUMMARY OF THE INVENTION

A composition for treating diseases in animals and plants includes at least one complex cation and inorganic coordination complexes formed by the coordinate bond formation between an electropositive mineral cation (positive) and molecular groups that pose unshared electron pairs.

The formulation as manufactured provides a delivery system for moving the mineral ions to the target disease area in plants and animals uses highly bioavailable cations through the complex ligand system. In some cases a salicylic acid preparation will become a source of enhanced transport of minerals that form an artificial super-oxide dismutase (SOD) radical that enhances the transport of free metallic ions, especially Zinc and Copper, to regions of the body for treatment of diseases such as arthritis and other bone and joint diseases. However, it must be stressed that all minerals discussed can be introduced to the target cells using the ligand system delivery system without the aid of additional compositions. The bioavailable minerals (BAMs) are highly effective, but the use of botanicals, Vitamin and Mineral supplements, urea formulations, and formulation additives improve the performance of the BAMs alone.

The composition and delivery mechanism may be used to treat a myriad of human and animal disease conditions including pain and cancer. Moreover, the composition and delivery system may aid human nutrition related problems as well as the general well-being of persons on a daily basis taking into account factors such as, sex, stress factors, genetics and the environment.

Additionally, the composition may take a plurality of different formulations each having a different bioavailable cation in the same or different proportions. Moreover, there are different mechanisms for administering the new products to ensure the bioavailable cation reaches the target area. For example, the composition may be introduced into the body of the animal or human by at least one of ingestion, injection, capsules, transdermal patches, cream or suave for use as a topical application. The principles described herein that successfully treat animal diseases can be applied against plant diseases as well by using the same chemistry and understanding of physiology as related to animals. The treatment of disease of plants is parallel to the treatment of animals, including humans in general principle using the technology discussed. The use of the bioavailability of the product can also be used in new fertilizers and use in hydroponics.

A composition for treating plant and animal diseases includes a therapeutically effective amount of an ionic mineral complex and a pharmaceutically acceptable carrier. The ionic mineral complex is an ionic mineral cation ligand bonded to a plurality of ammonia molecules enabling transport of said ionic mineral complex through a biological system to a target cell affected by a disease. The cation is at least one of Zinc, Silver, Manganese, Copper, Magnesium, Cobalt, Chromium, Molybdenum, Selenium, Vanadium and all other forms of these minerals.

A method for producing a composition for treating plant and animal diseases includes the activities of adding ingredients of Ammonium Hydrogen Sulfate (NH4HSO4), at least one mineral composition and distilled water to a mixture, agitating said mixture and diluting said mixture to a desired concentration. The activity of agitating is slow agitation process performed at a speed able to reduce exothermic interaction between the ingredients. The diluted mixture includes at least one ionic mineral complex encapsulated by ligand bound ammonia molecules. The diluted mixture may be combined with a pharmaceutically accepted carrier for delivery to a target cell.

### DETAILED DESCRIPTION OF THE INVENTION

The invention is related to a chemical composition for mitigating plant and animal diseases, and more specifically, to a composition and use of bioactive elements that are able to interrupt the metabolic pathway of various diseases affecting plants and animals.

### Minerals

The Kreb's cycle describes the metabolic pathways of the higher plants and animals where the parallel use of available fuels, oxygen, water, and other essentials to life in an aerobic world follow the same basic metabolism of the sugars, fats and proteins to form a structure. The basic oxygen driven metabolism drives the continuation of both the higher plants and animals. The metabolic system is a well-documented and familiar process involving the basic steps to produce a “higher” form of life. The protection provided by at least thirty-two steps in the process provides a system that protects the oxygen driven plants and animals. The higher forms of life exist and the basic knowledge we are incorporating in this patent can defeat lower order organisms, including diseases because the diseases follow a different metabolic pathway.

Lower order organisms are not near as complex regarding the metabolic pathway as the higher order organisms such as a tree or a human. The cycle followed by the lower order diseases such as bacteria and fungi follow a less complicated process that allows the disease to multiply at an almost exponential rate based on available resources in an anaerobic cycle that has far fewer steps. The disease utilizes all available minerals, sugars, fats and proteins to fuel the reproduction of cells using the abbreviated anaerobic cycle and the higher the rate of replication, the less oxygen available for healthy tissues surrounding the disease. Reduced oxygen aids in the fermentation process that is part of the favorable conditions required for the exponential growth of all organisms using the anaerobic cycle, including cancer.

Diseases affecting both plants and animals have an advantage because of the ability for exponential growth because of the shorter metabolic pathway versus the Krebs cycle. The composition exploits the flaw in the exponential expansion of the disease organism. Specifically, the flaw in lower organism metabolism is exploited by using the mineral gathering mechanism of the organism against itself. Lower orders of disease organisms gather the necessary minerals and other building blocks in an amount proportional to their availability in the environment. This differs from the metabolic processes of higher organisms that only gather enough of the elements and building blocks to satisfy the requirements of the Kreb cycle.

Thus, higher organisms will only incorporate minerals, etc at a rate necessary for survival and lower organisms, including diseases, will accumulate minerals in an amount that is toxic. Therefore, providing a high concentration of ionic minerals to a disease area would result in a toxic level of the mineral to disease organisms and allow survival of higher organisms so long as the dosage rate of the ionic minerals is below the toxic level for the higher organism. Killing the disease organism can occur at proper dosages of the ionic minerals while the higher order plants and animals would only gather the amount of mineral necessary to complete the Krebs Cycle and reject the excess minerals. There is a toxic level for even higher order organisms, but the amount of the minerals can be calculated to destroy disease organisms with little to no effect on the animal or plant.

The use of highly bio-available minerals (BAMS) at a rate that will kill the lower organism without impairing the function of the higher organism provides the active complex of the composition. As used hereinafter, all bioavailable minerals will be denoted as BAMS, and the designation for each mineral will be followed with a “B”. The ligand system transports all the minerals to the target areas in the plant or animal. The ligand system can be enhanced by the use of a few bioavailable minerals that can be incorporated into a salicylic acid complex that produces an artificial super-oxide dismutase cycle and transported quickly through an extra-cellular process (especially Copper and Zinc) to cells. The composition introduces the artificial SOD to a diseased tissue where peroxide is produced (an oxygen production process) that will also aid in killing disease cells, such as cancer. The creation of the artificial SOD cycle causes the disease causing organism to uptake an amount of ionic mineral that is toxic which results in death of the disease organism. Additionally, diseases such as cancer follow an anaerobic fermentation process and oxygen will destroy the anaerobic fermentation process thereby providing a secondary mechanism for destroying the disease. The composition may also contain sulfur that further aid in destroying cancer and aid in the relief of pain caused by arthritis and many other diseases and lead to the reversal of that disease. However, the prime mode of action is the minerals in a highly biologically available formulation carried by the ligand and assisted by other processes

Other mineral formulations are not as bio-available, and cannot pass through cellular tissues in the manner demonstrated by the chemical formulations described herein. In addition to the BAMS, the composition may include predetermined amounts of at least one of vitamins and botanicals. The vitamins and/or botanicals provide an extra measure for successful treatment of a disease or insuring well being of the animal (including humans).

The general principle of the formulation is rapid entry into the aerobic biological system of a plant or animal using a ligand carrier in an ionic form that penetrates and migrates toward an anaerobic disease system, if present. The product is capable of penetrating the barrier zone between the aerobic and anaerobic tissues if the disease is internal and usually encapsulated by a barrier consisting of mucous and a bacteria or virus. The unique quality of the formulation is the penetration of the membrane and the movement of large amounts of the mineral into the disease. The accumulation of the large amounts of mineral becomes toxic to the anaerobic system and the disease causing organisms or cells are terminated.

Plants are subject to the same general principals pertaining to the Krebs cycle. Again, the composition penetrates the membrane of the disease more readily than conventional preparations of minerals and the disease accumulates the mineral(s) to a toxic level, while surrounding healthy plant tissues are unaffected except at extremely high dosages.

Diseases have three vulnerable sites that can be attacked by treatment using the compositions and methods described using the ligand system protecting the ionic mineral:
1. Penetrating the outer membrane of the disease,
2. Destroying the internal components that drive the cells metabolism, and
3. Destroying the gene pool that may provide a future defense (resistance) against the introduced substance.

Minerals that are not in the bioavailable form will not be able to eliminate or otherwise disable the disease cells because the minerals cannot pass through the membrane coating the outer surface of the disease and/or cannot travel extra-cellular. The bioavailable minerals (BAM) will attack all of the diseases vulnerable targets in the cells because of the systemic capabilities of the formulation. The cell membrane is easily transversed and possibly ruptured, the inner cell is compromised because of the Kreb's cycle (aerobic vs. anaerobic) as described above and the genetic code of the animal and/or plant disease is destroyed. There will be no further deviations from the genetic code to produce new strains that may be resistant to the product. In fact, there are no known resistances to plant and/or animal disease if the primary source of the product being used is a mineral.

### Mode of Operation

The composition uses ionic mineral complexes that are capable of penetrating through the body having little to no effect on normal cells while destroying mutant cells. Further, the ionic mineral complexes are capable of penetrating cell membranes at a rapid pace and are blocked from entry to normal cells because the Kreb cycle will not allow entry of an abnormally high concentration of the mineral and discharged as excessive or travel through the cells without disrupting normal cell functions. The action of the ligand or SOD provides for extra-cellular transport of the minerals to the disease where mineral toxicity to diseased cells occurs. The process of destruction of the diseased cells is an over load of minerals and in some cases the added effect of oxidation of the anaerobic disease fermentation process.

Normal cells will die as a result of excessive exposure to almost any mineral. The composition involves the use of an appropriate amount of the mineral in an ionic mineral complex form that will cause a toxic effect to the mutant cell while not reaching a toxic level in normal cells.

Another mode of action examined as the possible method of destruction of cancer and other diseases is the effect of the ionic mineral complex on bacteria or other pathogens or saprophytic organisms that surround and act to encapsulate the disease. High numbers of bacteria, etc. have been identified in the protective membrane and may be responsible for the membranes existence. The BAM ionic mineral complexes having anti-microbial properties and are freely moveable between and through cells are capable of reaching the interface between the healthy and diseased tissues and destroying the disease organisms that exist in the protective membrane for diseases such as cancer. In studies performed using the BAM composition, cancers are rejected from the body in a sheath-like mass. The formulation has penetrated the sheath and destroyed the bacteria, and dissolved the sheath. The interior of the sheath contains the disease (including cancer) and the natural immune system, if intact, will reject the mass of cells contained in the sheath which may include bacteria, virus, or other disease causing organism contained within in a mass of cells that can move from an internal area of the body through the epidermis. The sheath is the mass created by the accumulation of millions of white blood cells that accumulate around the destroyed diseased tissue that are separated from healthy tissue at the interface where the bacteria, virus, other disease organism was located. Once dead, the interface containing the dead diseased bacteria and white-blood cells is separated from the healthy tissue and the mass of tissue inside the sheath (cancer, etc) is expelled from the body, much like a splinter is expelled from the body.

### Additives to BAM Minerals

The complex cations and inorganic coordination complexes formed by coordinate bond formations will be used in combination with inerts will be used in a specific formulation that may include, but are not limited to additives such as surfactants, di-methyl sulfoxide (DSMO), urea-based compounds, detergents, hydroscopic compounds such as, but not limited to Cell-U-Wet, and/or other chemicals, some to aid in penetration through the skin and/or for other reasons necessary in manufacture of formulations for injection, tablets, transdermal patches, etc.

### Botanical Extracts and Isolations

Plants, plant extracts and isolations have been used for centuries to treat certain diseases in humans and promote general wellness. One major factor in the cure of diseases and wellness is boosting the immune system and, several plants are known to have ingredients proven to help in fighting or the prevention of disease. The mineral ion zinc (ZN BAM) can be used in coordination with certain plant extracts for boosting the immune system and combating certain diseases. For example, extracts may include any of the following but are not limited to: Graviola (Annona muricata), Bitter Melon (Momordica charantia), Esplanheira Santa (Maytenus illcifolia), Mullaca (Physolis angulata), Vasourinho (Scoparia dulcis), Mutamba (Guazuma ulmifolia), Suma (Pfaffia paniculata), Cat's Claw (Uncaria tomantosa), Blood Root (Sanguinaria canadensis). The quality of the botanical correlates to the performance of the plant extracts and a person trained in the art is necessary in most cases to provide a measure of success.

For example, not all Graviola trees will yield the same amount of the active ingredient from the leaves or fruit, and someone skilled in selection of the leaves from specific trees can provide a superior extract following a detailed recipe. For instance, only bottled water (not chlorinated) must be mixed with a specific surfactant before adding cinnamon and the plant material and cooked in stainless steel or glass for five days at 140-160 degrees F. and twice filtered before storage at room temperatures and the product must not be taken with co-enzyme Q10 (COQ10) which is a food supplement. The results are remarkable when formed as a composition with the BAMS ligand complex and other supplements including but not limited to vitamin and minerals.

### Vitamin and Mineral Supplements

The use of BAMS will greatly improve general health, but not all minerals can be processed into a BAM. Therefore mineral supplements will be necessary in traditional forms to meet minimum daily requirements when boosting the strength of the immune system. It is true that the daily allowances for both vitamins and minerals can be obtained through a healthy diet, but when an individuals immune system is compromised and they are facing cancer or some other major disease, supplementing the food intake is vital to the mitigation of the disease.

An example of a rigorous and effective regimen of vitamins and minerals for building the immune system and relieving pain from arthritis, treating sugar diabetes, providing a stable mental attitude, lowering dependence on prescription drugs, lowering blood pressure, fighting alcoholism and drug addiction and building sound muscles includes, but is not limited to the following:

| MORNING | | | EVENING | | |
|---|---|---|---|---|---|
| Beta Carotene (Vit. A) | 25,000 | I.U. | Asprin (If Tolerant)—small | | |
| Vit. B-6 | 100 | mg | Zinc Gluconate | 250 | mg |
| COQ10 (Except | 100 | mg | Magnesium Sterate | 25 0 | mg |
| with Graviola) | | | Calcium Citrate | 600 | mg |
| Folic Acid | 800 | mg | Omega 3 (Fish Oil) | 1000 | mg |
| Vit. D | 400 | I.U. | Red Yeast Rice | 600 | mg |
| Selenium (Yeast) | 200 | mcg | Vit. E | 400 | I.U |
| Biotin | 1000 | mcg | Ester Vit. C | 1000 | mg |
| Chromium Piconate | 200 | mcg | For Joints) Gluco- | 500 | mg |

| | | | |
|---|---|---|---|
| Multi-Vitamin (Organic) that | samine Sulfate | | |
| contains Micro-Nutrients | Chondrotin Sulfate | 500 | mg |
| (Trace Elements) | MSM | 200 | mg |

(MSM—methlysulfonylmethane)

Boosting the immune system using a combination of botanicals, vitamins and minerals in combination with BAMS acts synergistically to provide the individuals-(bodies) own immune system to reject cancer. The BAMS provide a direct attack on the membrane sheath surrounding the cancer cells and a direct attack on the cancer itself by over-loading the cancer cells with minerals that become toxic to the cancer while not affecting the surrounding healthy tissues.

### Urea

Urea may be combined with any of the BAMS and plant extracts, Vitamins and Minerals as described above for enhancing the effect of a treatment and/or to promote wellness in humans. For example, urea breaks down the hydrophobic bonds of cancer cells when injected into and/or around cancers. The watery structure surrounding the cancer breaks and the cancer cells are unable to feed, and the cells become unprotected and exposed to attack by the body's immune system. The immune system then attacks the cancer cells. Urea also forms ligand bonds much like the ammonia that aid in increasing the bioavailability of the minerals. The addition of the urea in the formulation provides for a two-prong attack, and may also be a factor in transporting the cations as ligand bonds throughout the system.

The treatment of diseases such as cancers, for example, skin, colon, liver, prostate cancer will require different combinations of the ionic mineral complexes formulated with certain inerts and made for each specific application. Such formulations may include a product for the purpose of detection of cancer, for the use as a vaccination for cancer in warm-blooded animals including humans, pets or other animals and/or other uses to be described and which is provided that the product(s) includes at least one therapeutically effective formulation as detailed below. The composition may also contain pharmaceutically acceptable carriers, diluents, cream bases, hydroscopic additives, detergents and/or other carriers required for the therapeutically effective dosage to travel to the specific target site for the intended purpose (detection of cancer, treatment of cancer and/or vaccination, etc.}. The composition may be therapeutically administered by at least one of topical, oral, parenteral or other methods of application, in a therapeutically effective form of the formulation. Other diseases that can be mitigated by use of ionic mineral complexes by themselves or in combinations of ionic mineral complexes derived from the method of disassociation of compounds include, but are not limited to arthritis or any other degenerative disease. The same systems of ionic minerals can be used for promoting wellness of an individual to promote disease resistance, increase energy, etc. Formulations for applying the active ingredient(s) include, but are not limited to the above inert ingredients.

The manufacturing process described below produces a complex having ammonia ligand bonds with specific cations. The cations are “encapsulated” by the ligand bonds and are protected from being immediately bonded with the first available negative ions thus enabling free movement between and within cells of the body. Each of the minerals will be processed in the acid-base solution, resulting in products that have a high acidity value, yet not being corrosive to living tissue. The product is highly corrosive to inert biological materials such as paper, cotton, etc. A high concentration of reactive Ammonia is also produced by the acid-base reaction. Complex cations and inorganic coordination complexes are formed that are able to “encapsulate” the cations in a relatively stable fashion and allow transport throughout the body.

### Chemistry

TABLE 1
Ammonium Sulfate and Sulfuric Acid

| | | |
|---|---|---|
| Definition of terms : | | |
| 1. Sulfuric acid | H₂SO₄ | molecular weight = 98.07 grams |
| 2. Ammonium sulfate | (NH₄)₂SO₄ | molecular weight = 132.23 grams |
| 3. Ammonium hydrogen | (NH₄)HSO₄ | molecular weight = 115.10 grams |
| sulfate ( ammonium | | |
| bisulfate ) | | |

The Sulfuric Acid and Ammonia are combined in the presence of water:

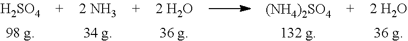

Ammonium sulfate in water has a typical pH of 5.5-6.0. Ammonium sulfate by itself when dissolved in water does not exhibit any ammonia odor. Solid ammonium sulfate can dissociate, losing one mole of ammonia and converting to ammonium hydrogen sulfate, NH₄HSO₄

(NH₄)₂SO₄→NH₃↑+NH₄HSO₄

In the presence of water, the ammonia lost can then recombine to;

NH₃+H₂O→NH₄OH or, (NH₄)⁺¹+(OH)⁺¹

The ammonium bisulfate, being a weakly acidic salt, does not react with the dilute and weak base, ammonium hydroxide to recombine into ammonium sulfate. Ammonium hydrogen sulfate is more acidic than ammonium sulfate. Neither a solution of ammonium sulfate nor ammonium bisulfate will burn the skin on contact. Additional loss of ammonia from solid ammonium hydrogen sulfate under conditions of high heat and moisture provides the following:

NH₄HSO₄+heat+moisture→NH₃+H₂SO₃+H₂O

The result is sulfurous acid. Sulfuric acid and ammonium hydrogen sulfate are a basic building block for producing the ligand bonded cation complex, hereinafter the ligand complex.
Ammonium hydrogen sulfate may be generated by two methods:
1. H₂SO₄+NH₄OH→NH₄HSO₄ (One mole of sulfuric acid and one mole of ammonium hydroxide (ammonia)
2. H₂SO₄+(NH₄)₂SO₄→2 NH₄HSO₄ (One mole of sulfuric acid, and one mole of ammonium sulfate)

The first reaction is very exothermic, and because the ammonia in water is not stable, ammonia fumes are generated, reducing the amount of ammonia available to react with the sulfuric acid. The result is a very acidic solution, having a small quantity of sulfuric acid unreacted due to the loss of some quantity of the ammonia in the steam generated by the high exothermic reaction. This is less desirable than the second reaction that is the preferred process used to produce the composition for treating plant and animal diseases. The physical and chemical properties of the ligand complex are shown in Table 2.

TABLE 2
PHYSICAL AND CHEMICAL CHARACTERISTICS

| | |
|---|---|
| Boiling Point: | 224.2 degrees F. |
| Vapor Pressure | 0.1 mm @ 68 degrees F. |
| Vapor Density | 1.00 (same as air) |
| Solubility in Water | Very soluble |
| Appearance and Odor | Clear liquid/mild odor |
| Specific Gravity | 1.35 |
| Melting Point | N/A |
| Evaporation Rate | N/A |
| Flash Point | None |
| Flammable Limits | None |
| Stability | Stable |
| pH | Less than 1.0 |

### Complex Ion Formation/Ligand Bonds

The Complex ions and inorganic coordination complexes are formed by the coordinate bond formation between an electropositive mineral cation (positive) and molecular groups that possesses unshared electron pairs (ammonia). Every metal ion has at least one coordination sphere which determines the number of coordinate bonds possible for each mineral atom. The coordinate bonds attract negatively charged ions possessing unshared electron pairs. All cations except Groups IA and IIA (periodic table) exist as complex cations with a definite number of coordinating groups bound to them. The cations use the unshared pair in attempting to fill gaps in the outer electron orbitals where those electron shells are incomplete. The bonds formed between the cation and the unshared pair of electrons are ligand bonds.

An exemplary compound produced as a result of the acid-base reaction when sulfuric acid is combined with ammonia sulfate described above is Ammonia (NH₃), is one of the products formed in the acid-base reaction. Ammonia is one of the compounds having an unshared pair of electrons that enables ligand bond formation between itself and the free cation in solution. The Nitrogen molecule of the Ammonia includes an unshared pair of electrons. Ammonia is very reactive in ligand bonding due to its respectively small size, and the unshared pair of electrons. The three hydrogen atoms cannot equalize the charge due to repulsion between the electron pair making ammonia polar. Therefore, for example, Ammonia may enter into the following examples of complexes:

1. Ag+ (Silver ion) Ag++2NH3→[Ag(NH3)2]+
2. Cu+2 (Copper ion) Cu+2+4NH3→[Cu(NH3)4]+2
3. Zn+2 (Zinc ion) Zn+2+4NH3→[Zn(NH3)4]+2
The number of ammonia molecules is double the metallic ion valence, and that the valence charge does not change. The unshared pair of electrons forms the ligand bonds, the ligand supplying both the unshared electrons. The resulting compound is a plurality of ammonia molecules ligand bonded to a single molecule of ionic Zinc forming a “BAM”, (an encapsulated mineral surrounded by ammonia “ligand bonds”). This molecular diagram is shown for purpose of example only and the Zinc cation may be substituted by any of the cation ions shown in Table 3.

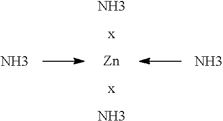

This compound including ammonia encapsulating a bioactive mineral cation is hereinafter referred to as a ligand complex. Additionally, urea may be included in the formulation results in ligand bonding of the cations with the urea. In this composition, the BAM minerals could be formulated with the urea producing a cream containing higher than anticipated mineral content than is normally expected in the formulations.
Examples of the ligand compounds may include, but are not limited to the following complexes shown in Table 3:

TABLE 3
Ion-Complexes with Ammonia (Ligand Complex)

| | | |
|---|---|---|
| 1. | Zinc | Zn[(NH₃)₄]⁺² |
| 2. | Silver | Ag[(NH₃)₂]⁺¹ |
| 3. | Manganese | Mn[(NH₃)₄]⁺² |
| 4. | Copper | Cu[(NH₃)₄]⁺² |
| 5. | Magnesium | Mg[(NH₃)₄]⁺² |
| 6. | Cobalt | Co[(NH₃)₄]⁺² → Co[(NH₃)₆]⁺³ + 1 e'* |
| 7. | Chromium | Cr[(NH₃)₆]⁺³ |
| | tri-valent | |
| | hexa-valent | Cr[(NH₃)₁₂]⁺⁶ |
| 8. | Molybdenum | Mo[(NH₃)₆]⁺³ |
| 9. | Selenium | Se[(NH₃)₈]⁺⁴ |
| | | Se[(NH₃)₁₂]⁺⁶ |
| 10. | Vanadium | V[(NH₃)₁₀]⁺⁵ |

*Normally, in aqueous solutions without complexing agents, Cobalt ⁺² is the favored state. In the presence of complexing agents such as ammonia, NH₃, complexes of Cobalt ⁺³ have greater stability, and are more stable in basic than acid media.

Additionally, ligand bonding is conducive to maintain the abundance of hydrogen ions. The resulting solution has a very low pH (at or near zero) reading because of the ligand bonded mineral ions. The product does not act as a conventional acid because of the stability of the mixture of ligands. The pH of the products are not indicative of the expected acid characteristics one might imagine at a pH of 1.0 or below. The solution can only be reduced by non-heat evaporation to a certain volume.

It is possible to mix the various minerals described in Table 3 according to the process described in U.S. Pat. No. 7,163,709 which is co-owned by Applicant and incorporated herein by reference because all are positively charged ligand complexes act to repel one another.

### Secondary Active Complex

Additionally, the chemical reactions described above result in the mixture including a high level of Sulfur which functions as a secondary active complex that operates synergistically with the ligand complex described above by accelerating the treatment of the affected cells. The high level of free sulfur is able to be transported to various locations in the biological system by normal mode of transport and works to speed reconstitution of damaged tissue affected by the disease. Sulfur is a non-metallic acidic macromineral usually consumed as part of a larger compound (Zinc, Copper, etc) and is not usually expressed as an aid to the mitigation of disease. However, the benefits of Sulfur are well known and the formulation that combines a high-Sulfur content (NH4HSO4) base with the minerals with radicals that contain Sulfur (Zinc Sulfate, etc) will provide an abundance of free Sulfur that accumulates in those regions of the human body that require attention on an as needed basis to maintain the system or to combat diseases. The preferred mineral radical would be a sulfate for that reason but is not necessary for the success of the free sulfur. The benefits of Sulfur include boosting the immune system and providing pain relief to targeted cells. The mechanism by which free Sulfur produced in this mixture operate similarly to the operative mode of Glucosamine Sulfate, Chondrotin Sulfate and MSM (methylsulfonylmethane).

### Method of Manufacture

An exemplary method of producing the composition having a ligand complex is described herein. Ammonium Hydrogen Sulfate (NH4HSO4) is prepared as described above and is used in the concentrated form. The Ammonium Hydrogen Sulfate is added to distilled water. The minerals added which are dissociated to become the bioavailable ionic mineral ligand complex are added in their respective sulfate forms, i.e. Zinc Sulfate, Copper Sulfate, Magnesium Sulfate, etc. Any of the minerals listed above in Table 3 may, in their sulfate form be mixed with the Ammonium Hydrogen Sulfate and distilled water to produce the ligand complex. Preferably, the mineral sulfate compound is manufacturing or pharmaceutical grade with no mineral impurities. The BAM Product(s) destined for utilization as injectables should be pharma grade with no impurities. The processing of the minerals to form BAMS is only a general guideline, and the concentration of the minerals in the solution can be adjusted to produce a more concentrated formulation and the exemplary composition has a reduced amount of mineral content.

An exemplary composition for making one gallon of the ten (10) mineral complexes listed above involves preparing a mix, under agitation, during a slow combination (to prevent a exothermic reaction) of the following ingredients:
98 ounces of water (H2O)
14 ounces of Ammonium Hydrogen Sulfate (NH4HSO4)
16 ounces (one of the ten listed) alone or in combination(s) with other minerals on the list to make a total of 16 ounces of mineral.
   The exemplary composition may include, for example, 16 ounces of Zinc Sulfate, 8 ounces of Zinc Sulfate and 8 ounces of Copper Sulfate; or 8 ounces of Zinc Sulfate, 4 ounces of Copper Sulfate and 4 ounces of Magnesium Sulfate. Any sulfate form of the minerals listed in Table 3 above may be combined in any ratio. The composition may include any number of mineral sulfate complexes in order to produce a composition having a number of ligand complexes equal to the number of different minerals in the mineral sulfate complexes added during mixing. The description of the mineral composition being in the sulfate form is for purposes of example only and any other form of the mineral composition including, but not limited to Oxides, Gluconates, Yeasts, Picolates, etc may also be used to produce the composition.

Each of the positively charged mineral complexes also act independently, much as a gas behaves in Dalton's Law. A solution containing a mix of the prepared minerals will act independent of one another and be assimilated by the body on an as-needed basis. For example, copper and zinc can be antagonistic to one another in an animal, but the two minerals can be put into the BAM formulation at the right proportions and counter-act the influence of one against the other. The Copper and Zinc also act to counter-balance each other in the physiologic on a basis of 7:2 (Zinc to Copper) When the BAM formulation in the form of ligand complexes contacts living tissue, a physiological change can occur where the minerals are un-encapsulated from the ligand complex and are useable as part of the metabolic pathway in the that organism.

The ligand formation is obtained during manufacture. However, the use of the ionic mineral in the formulation can be utilized to form an artificial SOD when a salysilic acid is added. An example of incorporation of a BAM as described with reference to the super-oxide dismutase (SOD) cycle will require copper (Cu) or zinc (Zn) or both minerals. The BAM's will be incorporated into the SOD on a as-needed basis (attach to the mineral complex) to make a Cu-SOD, a Zn-SOD, or a Cu,Zn-SOD. The enzyme SUPER-OXIDE DISMUTASE (SOD) catalyzes the dismutation of super-oxide into oxygen and hydrogen peroxide. Therefore, it is an important antioxidant defense in almost all cells exposed to oxygen. The SOD-catalyzed dismutation of super-oxide may be written using the following half-reactions:

M⁽ⁿ⁺¹⁾⁺−SOD+O₂⁻→Mⁿ⁺−SOD+O₂

Mⁿ⁺−SOD+O₂⁻+2H⁺→M⁽ⁿ⁺¹⁾⁺−SOD+H₂O₂.
where M may be, but is not limited to:
(a) Cu(n=1); (b) Zn(n=2); (c) Mn(n=2); (d) Fe(n=2); (e) Ni(n=2).

In this reaction the oxidation state of the metal cation oscillates between n and n+1. Several common forms of SOD exist and are proteins co-factored with Copper (Cu) and Zinc (Zn), or Manganese (Mn), Iron (Fe) or Nickle (Ni). Cytosols of almost all eukaryotic cells contain an SOD enzyme with Copper and Zinc (Cu—Zn-SOD)'s. The Cu—Zn-SOD (enzyme) is a homodimer of molecular weight of approximately 32,500. The Cu and Zn are joined primarily by hydrophobic and electrostatic interactions. The ligand complexes of copper and zinc are histidine side chains whereas the ligand complexes of the manganese ions are 3 histidine side chains. The incorporation of the ligand complex into the SOD enables the ligand complex to travel throughout the body in a protected form and without comprising the effectiveness of the ligand complex. Once the SOD with the ligand complex reaches a target cell, the cation within the ligand complex is released into the cell thereby overloading the target cell with the elemental cation and causing the cell to cease normal operation and function.

The resulting formulation can be prepared in many ways for application, and will vary with the intended use. Formulations prepared for treatment of a disease such as cancer may vary from products designed for wellness. In any case the formulations have been stable at a pH near or below pH 1.0 in a wide variety of carriers. However, the active composition is prepared using the liquid. Additionally, the low pH of the composition may be diminished if the composition crystallizes and may lose its efficacy.

The mineral cation ingredients in the active composition will vary in proportion depending on the intended use and be added in certain formulations depending on the type and purpose thereof. The added inerts used in the formulation will also vary considerably depending on the site of the application (skin surface, subcutaneous, interperneal, transdermal, inter-muscular, venous, intravenous solution, stomach or intestines, prostate, liver, kidneys, spleen, etc.) Other active compounds may be added if the proposed component proves to be beneficial to the formulation. However, the basic ingredients here shown are known to be efficacious without any additional components.

Additional elements may be included in the composition that aid in the overall effectiveness of the composition. For example, plant extracts such as botanicals may be incorporated into the composition. Gravila may be included in order to promote general immune system health that aid the body in fighting off the cancerous tissue that has been exposed due to the introduction of the high bioavailable ionic mineral as described above.

### Methods of Application of BAM Minerals to Target Sites

When describing a composition including a bioavalable ionic compound such as those listed above in Table 3, the composition may be denoted as its chemical abbreviation of the letter(s) for the mineral followed by the letter “P” (product) indicating that the composition is the product composition for treating a target site in a plant or animal. For example, the chemical composition having highly bioavailable Zinc encapsulated therein is written as “ZNP”.

Treatment of diseases and/or applications for the purpose of promoting general wellness may vary and will be, but not limited to be administered primarily as a topical application, taken orally, given by injection, transdermal patch, implant or by the use of other methods of treatment discussed such as application of the formulation through, or incorporated into and/or with a diluent or as a gel cap, tablet, powder, food additive, drops, liquid, beverage, rinse, mouthwash, gargle, pill, capsule, lozenge, cough drop, transdermal patch, ointment, salve, cream lotion, gel, intervenous drip, and/or adapted for periodical administrating a therapeutically effective dosage at least one of topically, orally, parenterally, intravenously or subcutaneously. The ligand complex composition has a low pH (less than 1.0) and, in order to remain effective, the low pH must be maintained when forming any of the above formulations.

The composition, depending on the type and concentration of the BAM maybe formed as a hemotherapeutic composition of the components will be used for treatment of blood diseases and/or for general wellness. When formulated as a homeotherapuetic composition, the composition may be formed in any of the manners for delivery or introduction into a plant or animal body system. The delivery mechanism may be in a form selected from a group of pills, powders, granules, tablets, micro-capsules gelcaps, nutritional supplements, liquids, drops, juices, beverages, food or drink additives, rinses, mouthwashes, solutions (including intravenous), and adapted for periodically administration of a therapeutically effective dosage of at least one of topically, orally, nasally, parenterally intravenously, subcutaneously, and all other methods of application.

### Example 1

### Bioavailable Zinc (ZN)

We will use cancer as an example of how this composition can be used to detect and/or cure this disease. The complex ion and/or inorganic coordination complexes using zinc can be prepared by combing and agitating zinc sulfate (ZnSO4), with a composition having the chemical formula (NH4HSO4 and water. The result is a zinc ligand complex that has the ability to penetrate through cell membranes without being blocked. The complex has a strong positive charge and is readily accepted into the cell. Other products containing zinc only produce a limited amount of ionic zinc. Zinc oxide, for example, releases zinc ion depending on the acidity of the product. The efficiency of the product was in direct relation to the amount of free zinc ions. Zinc Sulphate has a weaker bonding structure than that associated with Zinc Oxide and therefore, when combined as described above, the Zinc disassociates from the sulfate in the presence of the composition having the chemical formula (NH4HSO4 and becomes more bioavailable and more readily forms the ligand complex. Zinc Sulphate, for example, generates complex cations and inorganic coordination complexes that allow the ions to act independent without a counter-balance at a cell membrane and can be translocated throughout the body passing through healthy cells with no affect. Zinc Sulphate without being processed using the described chemistry will be rejected at the cell membrane and only a small amount of Zinc ions will enter the cell making the product highly ineffective.

Normal cell development is a structured system involving the incorporation of the required “building-blocks” consisting of minerals, etc. to form and maintain new cells. Excess minerals, etc. are either blocked from entry into the cell or expelled when in excessive quantities in the cell interior under normal conditions. Cell developments that are mutations may occur naturally or artificially. Mutated cell development does not follow the same developmental process as a normal cell. The amount of energy required to reproduce cancer cells is many times that of normal new cells. This large amount of energy required by the cancer cells is tied to the inefficient building of cells and many mistakes occur in the reproduction of the mutant cell in the accumulation of minerals etc., during cell formation. The active composition including the ligand complex uses the abnormal mineral intake of the mutant cells to overwhelm and destroy the cells by introducing a high level of high bioavailable zinc which disrupt the mutant cell formation.

An electropositive zinc cation complex will be accepted by the cancer cell in toxic levels thereby increasing the concentration of Zinc in the cell to toxic levels, causing the destruction of the mutant cells. Normal adjacent cells will expel the excessive zinc. Other minerals such as Copper (Cu) and Silver (Ag) and/or combinations of minerals prepared according to the invention are also effective. The composition delivers necessary minerals in ionic form into a mutant cell at levels that are toxic, causing destruction of the mutant cell while leaving normally developing cells intact. Other diseases of humans and other animals follow the same principal and other mineral(s) known to be effective in treating those conditions may be incorporated into a ligand complex and delivered to the diseased cell.

The BAMS will be formulated for the mitigation of specific diseases based on an evaluation of the animal (including humans) symptoms and diagnosis and be dispensed based on the age, gender, weight, etc of that individual. The method of application will vary based on the situation, but can include, but not be limited to injection, creams, salves, trans-dermal patch, ingestion, inhalation, etc.

Mitigation includes the prevention of diseases, and includes the use of the above methods of administering doses of the BAMS with other supplements such as vitamins and botanicals to provide a healthy body. It is important to understand that the BAMS can act independently, but for the best results in the most difficult cases include the use of vitamins and botanicals for support in the mitigation effort. For example, the BAMS can eliminate skin cancers, but the use of high doses of vitamin C (1000 up to 10,000 mg) are recommended along with other vitamins to provide an integrated internal defense against further spread. For example, botanicals such as Graviola are also recommended.

It is important to build the immune system. The BAMS work because the disease is exposed to the animal (including humans) system and the body can then eliminate the disease. Treatments with radiation and chemotherapy destroy the immune system, removing the only real defense the body has against cancer. The treatments do kill cancer, but the fine line between the elimination of the cancer and destruction of the immune system becomes critical, especially regarding individuals whose immune system has been challenged to a large extent before. The BAMS maintain and boost the immune system while destroying the disease. Other immune reinforcements should also be used in conjunction with the BAMS.

BAMS are also important in the normal physiology, including digestion, brain function, physical exertion, etc. and performance of the minerals is dependent on the synergistic relationship with vitamins and botanicals. Vitamins act in a synergistic fashion and are not absolutely necessary, but are used in a support mode. Botanicals also support the action of the BAMS, along with amino acids. An integrated system of BAMS, vitamins, botanicals and amino acids are large part in the total health of an individual along with exercise, rest, etc.

### Treatment of Plant Diseases

Diseases of plants are also subject to the same basic principles as diseases of animals. The amount of mineral concentration must be high enough to destroy the disease without causing phytotoxicity to the plant. There are several well-documented uses of minerals for the control of diseases on plants, including Zinc, Copper, Zinc/Copper, Silver/Copper and/or Zinc and Manganese formulations. There are several possibilities using the technology. There are several metallic compounds registered at the Environmental Protection Agency (EPA) for use against plant diseases. The inventor is proposing the use of several minerals and combination of minerals, including, but not limited to Copper/Zinc, Manganese/Zinc, etc in the BAM formulations. The principles applied to animals are the same for plants, since the Krebs cycle also applies to the life cycle of higher plants. Diseases such as bacteria and fungi follow an abbreviated short cycle and accumulate toxic levels of BAMS and die. The same type of SOD oxygenation cycle also occurs in plants, causing the anaerobic disease fermentation system to fail when the oxygenated BAMS are introduced.

Plants also require an “immune” system boost with the addition of auxins, microrhiza, to ward off diseases or to over come a disease-stressed condition. The use of BAMS in the proper proportions for each species of plants needed in a hydroponic system is in the micro-gram scale where other mineral additives would require a chemical that would leave a large residue of inert s associated with formulated product. The use of a foliar spray using the technology will provide a biologically active liquid nutrient and can be applied by ground or air using conventional application technology. The highly bioavailable minerals protect against diseases and can treat disease that is latent or active. Prop lactic treatments using the right combinations of minerals will not only prevent future diseases, but also provide the plant with the right requirements for necessary minerals. The other components of such applications may or may not include other additives include, but not limited to conventional fertilizers, auxins, gabbroic acid, microrhiza and other growth stimulation products, insecticides, matricides, other fungicides, etc that can be used in conjunction with a BAM treatment.

The use of diatomaceous earth (DE) with the BAM chemistry will provide the proper amounts of minerals (proportional) for most crops and act as an excellent foliar spray. All the components of the BAM formulation base (Ammonia Sulfate and Sulfuric Acid) and the DE are generally regarded as safe (GRAS) according to the Food and Drug Administration (FDA) allowing use on all food crops. The same formulation will protect the crop from not only diseases, but also insects due to the insecticidal characteristics of DE.

### Example 2

### Zinc

Elemental zinc accumulates in different portions of the body and is used on as needed basis by different bodily regions for general health and maintenance of the body. Additionally, zinc cations are utilized by the cells of the body to combat specific diseases.

A composition having highly bioavailable ionic zinc complexes may be produced from any of zinc sulfate, zinc chloride and/or other zinc compounds. Additionally, an exemplary composition may include other mineral ions and additives which enhance the efficacy of the treatment and have demonstrated the ability to detect and/or define the presence of diseases such as cancer, especially skin cancer in warm-blooded animals, especially man. The composition is produced in a manner described above and results in ligand complexes of Zinc that, once in the cell, may be more readily used by the various cellular processes thus resulting in a superior source of bioavaliable zinc.

### Topical Zinc Composition

An exemplary zinc composition may be produced to treat cancer, and more specifically, skin cancer. The composition may include a therapeutically effective formulation of the above components consisting of Zinc 7 parts: Copper 2 parts in a cream that will not interfere with the acidity of the product, with or without Vitamin A, C and E to form a zinc/copper ligand complex in a pharmaceutically acceptable carrier(s) or diluent(s) have demonstrated the ability to destroy skin cancer when the formulation is administered by topical application to the site of the cancer. Additionally, the composition may also include other ionized mineral complexes in combination with zinc ligand complex. Upon application thereof, cancerous cells die and are expelled and/or absorbed by the body within a period of as little as four days and usually within thirty days.

An exemplary method of applying the topical composition includes applying a topical composition to the infected area of the skin in a layer substantially 2-3 mm in thickness. The area having the composition is covered with a bandage or a transdermal patch prepared with a similar formulation. The treated area should not be disturbed for three to four days. After three to four days, remove the covering at that time being careful not to disturb the lesion at all especially by attempting to manually remove the infected section of skin. The cancer should fall out or be expelled from the host in time period ranging from five to twenty days from the original date of topical application. If the lesion is cancerous, a large scab will form or the cancer may be absorbed into the body. The body may also reject the cancer through the skin, forming a mass of puss and other fluids. If the lesion is not cancerous, there may be some skin irritation, but there will not be any significant reaction. If a scab appears, aloe juice may be applied onto the scab a few times daily until the scab falls off and the skin underneath begins to heal before another course of treatment. The other intervention tools include, but are not limited to, vitamins, including vitamin C at dosages approaching 10,000 mg/kg body weight and other vitamins to boost the immune system Vitamin A, B, C, D, E, and other vitamins, including, but not limited to Vitamin B-17 (Laetrille) that can oxidize the blood and relieve pain.

### Liquid Zinc Composition

An exemplary liquid zinc composition may be formed that includes a therapeutically effective formulation of a zinc ligand complex resulting in highly bioavailable zinc cations. Additionally, other ionic mineral complex, as will be discussed hereinbelow and which may be used to treat other conditions, may be including in the liquid zinc composition. The liquid zinc composition may be formulated so that the product composition may be administrated at least one of orally, as an injection, incorporated in a transdermal patch, via intravenous injection and any other pharmaceutically acceptable manner of introducing a composition into a plant and/or animal system. In the liquid form, the zinc composition may be able reach the internal organs that have cancerous growths. The liquid zinc composition may be used for the purpose of detecting and/or destroying cancers including, but not limited to stomach cancer, thyroid cancer, lymph node cancer, prostrate cancer, etc and possibly other diseases with a similar mode of action as cancer.

### Zinc Ligand Composition Vaccine

A vaccination including a therapeutically effective amount of a zinc ligand complex in conjunction with other elements known in the art of making vaccines may be formed and used to treat cancerous cells. Upon vaccination, the cancerous cells actively take in the active formulation of zinc ligand complex into the cancer cell. The active zinc ligand complex interrupts the cell cycle of the cancer cell resulting in a cytotoxic reaction and/or a chemical reaction expelling exposing the cancer to the known immune response system of the host. The process causes the activation of “T” cell antigens, which in turn form antibodies that also attack the cancerous tissue.

An exemplary composition has an amount of zinc ligand complex that results in an amount of zinc contained in the expelled cancerous tissue being in an amount of substantially 0.9%. The ligand complex, having a zinc cation in a highly bioavailable form, enables cells to take in an amount of zinc that is greater than three thousand times the normal level for zinc in cells. It is known that zinc interferes with the up-take of necessary minerals, including but not limited to copper, iron, selenium, etc. and also has a cytotoxic reaction at the levels detected in the analysis. The process of expelling the cancer is an immune response. Other methods of producing an immune response may be the result of the zinc ions only, but may also be the result of the combination of other ionized minerals. Vaccination of a person or other warm-blooded animal is possible using the latest vaccination technologies with the formulation of the composition.

A further vaccine composition may include the zinc ligand complex and any of dead or dying cancer cells that have been expelled from host, amino acids or protein that will induce an immune response. Introduction of this composition induces the formation of “T” cell antigens that form antibodies that will attack cancer cells which may become active in host in the future.

### Other Additives

Other ionized mineral ligand complexes derived using the manufacturing process specified above may be combined with the zinc ligand complex composition. These other ionized mineral ligand complexes may include any of copper, silver, gold selenium and manganese and combinations of such minerals that have similar actions against cancer and/or other diseases where swelling and irritation occur.

Additionally, ionic mineral ligand complexes manufactured using the Posilator process can be combined with plant extracts in certain cases where a cancer requires the use of such extracts. For example, plants in the Family Papaveraceae including the active component(s), such as an alkaloid or combinations of alkaloids and/or alcohol(s) that possess other anti-cancer properties that are used, or may be used in an anti-cancer drug such as in Sanguinaria canadensis. For example, Sanguinaria canadensis has numerous components, including Sanguinarine, a benzophenanthriidine alkaloid derived from the rhizomes. It is a catatonic molecule which converts from an iminium ion form at pH<6 to an alkanolamine form at pH>7. Sanguinarine extract is composed of sanguinarine and five other closely related alkaloids. Other chemical compounds such as the alcohols are also present in the root that may also be active and may not yet been specifically tested for anti-cancer effects. Any other plants in the family Paperveraceae may also include these specific compounds and may be included in a zinc ligand composition. Graviola is especially effect against cancers.

Additional inert ingredients will provide a base for the formulation and increase the effectiveness of the product(s) by aiding in the absorption of the active ingredients and in some cases provide an additive effect acting much as a synergist. Combinations of mineral BAMS may be used in any single therapeutic composition. For example, a composition including a predetermined amount of zinc ligand complexes with a predetermined amount of copper ligand complexes may be used in the treatment of cancer for the alleviation of pain and inflammation.

In addition, the invention may use ZNP generated through electrolysis, an ionization reaction and/or an ionic disassociation process with other active compounds including Sanguinaria canadensis, Kigelia africana, Larrea mexicana, and/or Annona muricata, Graviola and/or Tahehjua avellanedae in various amounts that produce results together that are not possible when only one component is used. The botanicals help build the immune system and allow the individual to ward off diseases. The immune system is the first line of defense against virus and bacterial diseases and supports any other intervention (medicine, etc) that is given a patient.

The BAMs directly attack the disease, such as cancer, and remove the sheath surrounding the cancerous tissue. The BAMs also are also absorbed in such large quantities by the cancer that toxic levels are reached and the cancer is destroyed. Any surrounding tissues are also attacked the same way. The immune system in the meantime is building white-blood cells that destroy any cancer cells in the vicinity and begin repair of the damaged tissues.

Clove oil and/or another natural or synthetic products may be used as a local anesthetic in the formulation, but may not be necessary if a local anesthetic is administered in cases involving application to the skin or where other sensitive organs are involved. Clove oil is a volatile oil that contains eugenol along with other components that act as a local anesthetic and also enhance the up-take of the active ingredients of the formulation, therefore a affecting the speed and efficiency of the formulation (synergetic reaction).

Other ingredients such as Amino Acids, Vitamins, hormones and other minerals may be included in the zinc ligand complex composition. Amino acids may include but are not limited to Picolinic Acid that facilitates the absorption of Zinc and Tryptophan which enhances the absorption of Zinc. Tryptophan is an essential precursor for the production of Picolinic Acid, which in turn greatly enhances the absorption of Zinc into the body's Cells. Hormones may include melatonin that increases the body's absorption and utilization of Zinc and enhances the beneficial effects of Zinc on the Thymus. Minerals may include Germanium that enhances the absorption of Zinc, Manganese which enhances the function of Zinc and Sulfur which facilitates the transportation of Zinc around the body. Vitamins may include Vitamin A that enhances the function of Zinc, Supplemental Vitamin B6 increases the intracellular concentration of Zinc (Vitamin B6 enhances the conversion of Tryptophan to Picolinic Acid) and Vitamin E which enhances the absorption of Zinc. These additional compounds are described for purpose of example only and any amino acid, vitamin, hormone or mineral that has a beneficial effect on bodily zinc processing may be incorporated into composition.

### Example 3

### Copper

A composition having a copper ligand complex may be formed in a manner described herein above with respect to Zinc. The copper ligand complex composition may be introduced into the biological system (i.e. human, plant and/or animal) to treat various diseases or conditions affecting the biological system. The copper ligand complex may be formed in at least one of a topical composition, a liquid composition or as a vaccine and may be produced in any form of delivery mechanism that has been previously specified.

An exemplary ligand complex composition having a copper ligand complex and zinc ligand complex in a ratio of 2:7 may be formed by combining the zinc and copper ligand complexes with the chemistry described above and water and agitating until mixed. The composition may be diluted to an effective amount and combined with an inert carrier ingredient enabling delivery of the copper/zinc ligand complex to a patient. An exemplary composition as described herein may be used to treat a patient having deficient levels of hemoglobin.

A composition including the copper ligand complex and Aspirin forms Copper Salicylate that has a high level of bioavailable copper. The Copper Salicylate has properties almost identical to those of Superoxide Dismutase. The highly effective anti-inflammatory agent has strong anti-viral properties and is a better anti-inflammatory than cortisone without the side effects. A deficiency of cytochrome oxidase is the main metabolic defect of cancer cells that causes a blockage of cellular respiration or oxidative energy production. Bio-available copper (CUP) and salicylate form a Super Oxide dismutase (SOD) and make copper available to the Cytochrome Oxide enzyme that is copper dependent. The process is similar to the use of an asprin to relieve pain where the salicylate combines with copper in the stomach lining and is transported to the site of the pain.

Additional elements may be added to a copper ligand complex composition for treating various diseases. These additional elements include, but are not limited to, Dehydroepiandrosterone (DHEA) which helps to prevent copper-induced lipid peroxidation by helping to prevent free radical damage to the body's Lipids caused by copper-initiated peroxidation processes; Boron and/or Cobalt which enhance the function of Copper and Iron which aids in the proper functioning of Copper within the host.

A composition including the copper ligand complex with other additives and/or other ligand complexes may be used to treat conditions of the cardiovascular system, digestive system, immune system, digestive systems, metabolic conditions, musculoskeletal system, nervous system.

### Example 4

### Magnesium

A composition having a magnesium ligand complex may be formed in a manner described herein above with respect to Zinc in Examples 1 and 2. The Magnesium ligand complex composition may be introduced into a biological system (i.e. human, plant and/or animal) to treat various diseases or conditions affecting the biological system. The magnesium ligand complex may be formed in at least one of a topical composition, a liquid composition or as a vaccine and may be produced in any form of delivery mechanism that has been previously specified.

A composition having a magnesium ligand complex alone or in conjunction with at least one other different ionic ligand complex may be used to treat conditions affecting any of the adrenal system, musculoskeletal system, the cardiovascular system, digestive system, excretory system, immune system, nervous system, respiratory system, and conditions relating to protein production. The composition including the magnesium ligand complex provides a highly bioavailable magnesium to target cells enabling treatment of various conditions that may be known to require additional levels of magnesium that the host does not possess.

Additional elements may be included in a composition having a magnesium ligand complex and include but are not limited to, vitamin A which enhances the function of Magnesium, vitamin B6 which increases the intracellular concentration of Magnesium, vitamin C which increases the body's absorption of Magnesium.

### Example 5

### Selenium

A composition having a selenium ligand complex may be formed in a manner described herein above with respect to Zinc in Examples 1 and 2. The selenium ligand complex composition may be introduced into a biological system (i.e. human, plant and/or animal) to treat various diseases or conditions affecting the biological system. The selenium ligand complex may be formed in at least one of a topical composition, a liquid composition or as a vaccine and may be produced in any form of delivery mechanism that has been previously specified.

A composition having a selenium ligand complex alone or in conjunction with at least one other different ionic ligand complex may be used to treat conditions affecting any of the adrenal system, musculoskeletal system, the cardiovascular system, digestive system, excretory system, immune system, nervous system, respiratory system, different systemic and organ based cancers and conditions relating to protein production. The composition including the magnesium ligand complex provides a highly bioavailable selenium to target cells enabling treatment of various conditions that may be known to require additional levels of selenium that the host does not possess.

Additional elements may be included within a composition having the selenium ligand complex that enhance the effectiveness thereof. These elements include but are not limited to, other ionic mineral ligand complexes, methionine which is increases the absorption, transport and bioavailability of selenium and is the most effective Amino Acid for chelating selenium supplements, taurine which facilitates the transport of Selenium into and out of the body's Cells, Sulfur which helps to transport Selenium around the body and vitamins including Vitamin E which can reduce the requirement for Selenium when Selenium is deficient

In addition to the Examples discussed above, other ionic mineral ligand complex compositions may be formed which include highly bioavailable levels of the particular mineral that are more readily delivered to and used by the cells in order to treat various conditions. Other minerals that may be formed into a ligand complex using the process described above include sulfur, manganese, chromium, molybdenum, cobalt, calcium, silver and vanadium.

The examples of different mineral ligand complexes described above may be formulated to treat various cancerous tissue of either human or animal hosts. Specifically, the effectiveness that the particular mineral has on the specific cancerous tissue is dependent on the physiological system affected by the cancer. Thus, the composition having an ionic mineral ligand complex may be used to detect cancerous tissues or treat malignant or pre-malignant tumors.

The composition including an ionic mineral ligand complex is applied in a thin layer to the suspect area. This example is for skin related cancers and assumes a topical application. However, application topical application as a thin layer is described for purpose of example only and application may be via ingestion and/or intravenous or subcutaneous injection into an affected area depending on the location or suspected location of the cancerous tissue. The treated area is covered but not confined for 24 hours. After 24 hours, the covering and any remaining composition are removed the area is to be kept clean and/or be covered to avoid additional contamination.

The area will react to the treatment according to the type of lesion present. Cancer will be detected when a strong reaction takes place at the suspect site. No pronounced reaction will take place if the lesion is not cancerous. If cancer is present, the reaction will be pronounced, with swelling and redness in the area.

The cancer lesion(s) treated will exhibit the same symptoms as the detection process. The swelling and redness will persist and the skin may develop a boil that may break and be sorbed into the body. The boil may be hard at first, but eventually become soft before breaking through the skin or being sorbed into the system. The process from treatment to the final disappearance may take from 10 to 20 days.

The ionic mineral ligand complex composition will detect skin cancers and other types of cancer and eliminate at the rate of 95% or better for skin cancers and have the potential for a high level of efficacy in other cancers. The composition activates the immune system of warm-blooded animals to “recognize” and destroy the cancer. This principal is the first form of an immune response. The result desired is that the animals immune system prompts the individual cells to recognize the cancer and to expel the cancer from the animals system through the skin, the bowls and/or the urine or be absorbed into the system.

The ligand complex are introduced into the cancer cell and the minerals dissociate from the complex and are highly bioavailable minerals that kill the cancer cells through the different modes of action causing degradation of the growth and death of the cancer cells, much like anti-bodies do to any foreign body during an immune response. Cancer is typically not recognized as a threat by the immune system of the body. The composition exposes the cancer as a foreign body to the immune system which attacks the cancer, activates the immune response causing the body to destroy the cancer and/or expel and/or absorb the cancer. Additionally, in animals without an immune system, the composition will not be able to promote immune response. However, the formulation has cytotoxic effects that will possibly kill the cancer in immune resistant animals.

Upon application of the composition, the cancerous tissue is expelled from the body in the form of a boil or cyst that breaks and pours forth the cancer in a sheath (mass), while not affecting non-malignant growths. This method of cancer detection is fast, easy to administer and near 100% effective. The additional benefit is that a 95% success rate has been realized in removing skin cancers, for example.

As discussed above, the composition may be administered topically by at least one of transdermal patch, ointment, salve, cream lotion, gel, solution and the like. The composition may be delivered by injection including venous injection and/or intravenous solutions into the cancer or cancer infected tissue and/or injected adjacent to the cancer or cancer-infected organ(s)). The composition may be administered orally or sublingually in at least one of gel cap, tablet, powder, food additive, food, drops, liquid, beverage, pill and/or capsule form to animals (including humans). Any oral administration may be formed as a time released formulation for internal cancerous tissues. The composition may be formulated as a mist able to be ingested by inhalation.

It will be understood that each of the elements described above, or two or more together may also find a useful application in other types of methods differing from the type described above.

Since the invention is described with reference to different embodiment and pointed out in the annexed claims, and since numerous modifications and changes may become readily apparent to those skilled in the art after reading this disclosure, it should be understood that we do not wish to limit the scope of the overall invention to the exact composition, or method of making same, described above and claimed below, since it will be understood that various omissions, modifications, substitutions and changes in the forms and details of the invention and in its application can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

I claim:

1. A composition for treating plant and animal diseases comprising a therapeutically effective amount of an ionic mineral complex and a pharmaceutically acceptable carrier.

2. A composition as recited in claim 1, wherein said ionic mineral complex comprises an ionic mineral ligand bonded to a plurality of ammonia molecules enabling transport of said ionic mineral complex through a biological system to a target cell affected by a disease.

3. A composition as recited in claim 1, wherein said ionic mineral complex is a cation and is at least one of Zinc, Silver, Manganese, Copper, Magnesium, Cobalt, Chromium, Molybdenum, Selenium and Vanadium.

4. A composition as recited in claim 1, further comprising at least one of surfactants, di-methyl sulfoxide (DSMO), urea-based compounds, detergents, hydroscopic compounds, and any other carrier for delivery of the active to the disease.

5. A composition as recited in claim 1, further comprising at least one plant extract selected from the group consisting of Graviola (Annona muricata), Bitter Melon (Momordica charantia), Esplanheira Santa (Maytenus illcifolia), Mullaca (Physolis angulata), Vasourinho (Scoparia dulcis), Mutamba (Guazuma ulmifolia), Suma (Pfaffia paniculata), Cat's Claw (Uncaria tomantosa), Blood Root (Sanguinaria canadensis).

6. The composition as recited in claim 1, wherein said composition is formed for at least one of topical application, oral ingestion, intravenous and subcutaneous injection, incorporation into transdermal patch, incorporation into a gel cap, tablet, powder, food additive, drops, liquid, beverage, rinse, mouthwash, gargle, pill, capsule, lozenge, ointment, salve, cream, lotion and gel.

7. The composition as recited in claim 1, wherein said composition may be introduced into a biological system by at least one of topical application, orally ingestion, parenterally, intravenously or subcutaneously.

8. The composition as recited in claim 1, wherein said composition has a pH of less than 1.0.

9. The composition as recited in claim 1, further comprising therapeutically effective amounts of two or more ionic mineral complexes and a pharmaceutical carrier, wherein said two or more ionic mineral complexes are in varying ratios, including a ratio of 1:1.

10. The composition as recited in claim 1, further comprising ionic zinc mineral complex and ionic copper mineral complex and pharmaceutical carrier, wherein the ration of Zinc to Copper is 7:2.

11. The composition as recited in claim 1, further comprising at least one of a mineral supplement and a vitamin supplement.

12. The composition as recited in claim 1, wherein upon introduction into a biological system, said composition bonds with a superoxide dismutase composition and transported throughout the biological system for delivery to said target cells.

13. The composition as recited in claim 1, wherein said ionic mineral complex includes a cation and, upon introduction into the target cell, said cation is dissociated from said ionic mineral complex and is taken up by a disease causing organism in an amount toxic to the disease causing organism causing death thereof without harming a host biological system.

14. The composition as recited in claim 1, wherein upon introduction to the target cell, said composition stimulates a host immune response by degrading a sheath around the target cell.

15. A method for producing a composition for treating plant and animal diseases comprising the activities of:
adding ingredients of Ammonium Hydrogen Sulfate (NH4HSO4), at least one mineral composition and distilled water to a mixture;
agitating said mixture; and
diluting said mixture to a desired concentration.

16. The method as recited in claim 15, wherein said activity of agitating is a slow agitation at a speed able to reduce exothermic interaction between the ingredients.

17. The method as recited in claim 15, wherein said diluted mixture includes at least one ionic mineral complex encapsulated by ligand bound ammonia molecules and,
further comprising the activity of combining the diluted mixture with a pharmaceutically accepted carrier.

18. The method as recited in claim 16, wherein said activity of combining with a pharmaceutically accepted carrier includes forming the composition suitable for at least one of topical application, orally ingestion, parenterally, intravenously or subcutaneously introduction into a biological system.

19. The method as recited in claim 15, wherein said activity of adding ingredients further comprises the activity of adding at least one of surfactants, di-methyl sulfoxide (DSMO), urea-based compounds, detergents and hydroscopic compounds and at least one botanical including any of Graviola (Annona muricata), Bitter Melon (Momordica charantia), Esplanheira Santa (Maytenus illcifolia), Mullaca (Physolis angulata), Vasourinho (Scoparia dulcis), Mutamba (Guazuma ulmifolia), Suma (Pfaffia paniculata), Cat's Claw (Uncaria tomantosa), Blood Root (Sanguinaria canadensis)

20. The method as recited in claim 15, wherein said at least one mineral composition includes any of zinc, Silver, Manganese, Copper, Magnesium, Cobalt, Chromium, Molybdenum, Selenium and Vanadium.
